(19) United States
(12) Patent Application Publication
(10) Pub. No.: US 2002/0136743 A1
(43) Pub. Date: Sep. 26, 2002

(54) COSMETIC COMPOSITIONS

(76) Inventors: ANNE LANGLOIS, MIDDLESEX (GB); YASMIN TINA WALLBANKS, BERKS (GB)

Correspondence Address: THE PROCTER & GAMBLE COMPANY, PATENT DIVISION, IVORYDALE TECHNICAL CENTER - BOX 474, 5299 SPRING GROVE AVENUE, CINCINNATI, OH, 45217, US

(21) Appl. No.: 09/180,921

(22) PCT Filed: May 16, 1997
(86) PCT No.: PCT/US97/08352

(30) Foreign Application Priority Data
May 17, 1996 (GB) 9610318.9
Sep. 12, 1996 (GB) 9619111.9

(51) Int. Cl. A61K 9/00; A61K 6/00

(52) U.S. Cl. 424/400

(57) ABSTRACT

A cosmetic composition- in the form of a waterinoil emulsion comprising: (a) continuous oil phase and. (b) discontinuous aqueous phase comprising (i) water; (ii) acidic skin care active which is insoluble in said aqueous phase; and (iii) nonionic surfaictanit selected from polyoxyethylenepolyoxypropylene ethers of C4C22 alcohols. aind mixtures thereof. The compositions of the invention provide increased product stability and improved acidic skin care active solubility.

### FIELD OF THE INVENTION

The present invention relates to cosmetic compositions and more particularly, to pigmented foundation make-up compositions and concealers.

### BACKGROUND OF THE INVENTION

A foundation composition can be applied to the face and other parts of the body to even skin tone and texture and to hide pores, imperfections, fine lines and the like. A foundation composition is also applied to moisturize the skin, to balance the oil level of the skin and to provide protection against the adverse effects of sunlight, wind and the harsh environment. Make-up compositions are generally available in the form of liquid or cream suspensions, emulsions, gels, pressed powders or anhydrous oil and wax compositions. Such cosmetic make-up compositions are described in U.S. Pat. No. A-3,444,291, U.S. Pat. No. A-4,486,405, U.S. Pat. No. A-4,804,532, U.S. Pat. No. A-3,978,207, U.S. Pat. No. A-4,659,562, U.S. Pat. No. A-5,143,722 and Nakamura et al., Preprints of the XlVth I.F.S.C.C. Congress, Barcelona, 1986, Vol. I, 51-63 (1986). Foundations in the form of water-in-oil emulsions are well known and provide good coverage and good skin feel, wear and appearance. At the same time, it would be desirable to provide a foundation composition having topical anti-acne activity. There are many compounds which are known to exhibit anti-acne properties when applied topically to the skin. A commonly used keratolytic agent having anti-acne activity is salicylic acid. As salicylic acid is virtually insoluble in water, it is difficult to incorporate it into the aqueous phase of an emulsion composition. Delivery of salicylic acid from the pigment-containing oil phase of an emulsion foundation composition can, however, lead to discolouration of the composition due to interaction between the salicylic acid and pigments, especially of the iron oxide type. It would therefore be desirable to deliver the salicylic acid in soluble from from the aqueous phase.

In addition, since acidic skin care agents are most active at low pH (when a high concentration of free acid is present in solution) it would be desirable to deliver the agent from an aqueous phase at a pH at which it exists significantly in protonated form.

Attempts have been made to incorporate salicylic acid into the aqueous phase of an emulsion composition. For example, WO 95/04517 discloses a make-up composition in the form of an emulsion comprising an acidic anti-acne active dissolved in the aqueous phase and a pigment or mixture of pigments dispersed in the oil phase. PVP is disclosed as a complexing agent for aiding the solubilisation of salicylic acid. However, it would be desirable to further improve the solubility of salicylic acid and other acidic skin care actives in the aqueous phase of emulsion compositions. Improving the solubility of active allows an increase in the level of water and a decrease in the level of alcoholic solvent in the aqueous phase. It is accordingly a primary object of this invention to provide a cosmetic composition comprising an aqueous solution of an acidic skin care active having improved solubility of active.

It is also an object of the invention to provide a cosmetic composition having improved product stability.

It is a fiurer object of the present invention to provide a cosmetic composition having improved oil control and improved appearance when applied to the skin.

### SUMMARY OF THE INVENTION

In accordance with one aspect of the present invention, there is provided a cosmetic composition in the form of a water-in-oil emulsion comprising:

(a) continuous oil phase and;

(b) discontinuous aqueous phase comprising:

(i) water

(ii) acidic skin care active which is insoluble in said aqueous phase; and

(iii) nonionic surfactant selected from polyoxyethylene-polyoxypropylene ethers of C4-C22 alcohols, and mixtures thereof

The cosmetic compositions of the present invention provide improved solubility of skin care actives together with improved product stability.

All levels and ratios are by weight of total composition, unless otherwise indicated. Chain lengths and degrees of alkoxylation are also specified on a weight average basis.

### DETAILED DESCRIPTION OF THE INVENTION

The cosmetic composition according to the present invention comprises an aqueous phase comprising an aqueous or hydroalcoholic solution of an acidic skin care active, an oil phase and a nonionic surfactant selected from polyoxyethylene-polyoxypropylene ethers of C4-C22 alcohols, and mixtures thereof. The composition is in the form of a water-in-oil emulsion.

### Acidic Skin Care Active

The compositions of the present invention comprise an acidic skin care active. As used herein the term acidic skin care active means any skin care active containing an acidic finctional group (e.g. carboxy, sulfonic), which is insoluble in the aqueous phase. The term “insoluble” as used herein means less than 0.2% of the acidic skin care active is luble in the aqueous phase at pH 3 and 25° C. It should be noted that the phrase “insoluble in the aqueous phase” as used herein means that the acidic skin care active is not soluble in the aqueous phase when the polyoxyethylene-polyoxypropylene ether of C4-C22 alcohol is not present in the aqueous phase. Suitable skin care actives can be selected from hydroxycarboxylic acids. The hydrocarboxylic acids for use herein can be selected from hydroxymonocarboxylic acids and hydroxydicarboxylic acids and mixtures thereof The hydroxycarboxylic acids may also be present as a free acid, lactone or salt form or as stereoisomers such as D, L, DL and meso forms. The lactone form could be either inter or intramolecular lactone, however, most common ones are intramolecular lactones with a ring structure formed by elimination of one or more water molecules between a hydroxy group and the carboxylic group. Since the hydroxycarboxylic acids are organic in nature, they may form a salt or a complex with an inorganic or organic base such as ammonium hydroxide, sodium or potassium hydroxide, or triethanolamine.

Suitable hydroxycarboxylic acids can be selected from hydroxymonocarboxylic acids having the following chemical structure:

R₁(CR₂OH)ₘ(CH₂)ₙCOOH

wherein R₁, R₂=H, alkyl, aralkyl or aryl group of saturated or unsaturated, straight or branched chain or cyclic form, having from 1 to 25 carbon atoms; m=1,2,3,4,5,6,7,8 or 9; n=2 or a numerical number up to 23; provided that when R₁=H, n=6 or a number up to 23.

Typical alkyl, aralkyl and aryl groups for R₁ and R₂ include methyl, ethyl, propyl, isopropyl, benzyl and phenyl. The hydrogen atoms of the R₁ and R₂ and (CH2)n may be substituted by a nonfunctional element such as F, Cl, Br, I, S or a radical such as a lower alkyl or alkoxy, saturated or unsaturated, having I to 9 carbon atoms. Representative hydroxymonocarboxylic acids are phenyl 2-hydroxyacetic acid (mandelic acid), phenyl 2-methyl 2-hydroxyacetic acid, 3-phenyl 2-hydroxypropanoic acid (phenyllactic acid), 2,3,4,5-tetrahydroxypentanoic acid, 2,3,4,5,6-pentahydroxyhexanoic acid, 2-hydroxydodecanoic acid (alpha hydroxylauric acid), 2,3,4,5,6,7-hexahydroxyheptanoic acid, diphenyl 2-hydroxyacetic acid (benzilic acid), 4-hydroxymandelic acid, 4-chloromandelic acid, 2-hydroxyhexanoic acid, 5-hydroxydodecanoic acid, 12-hydroxydodecanoic acid, 10-hydroxydecanoic acid, 16-hydroxyhexadecanoic acid, 2-hydroxy-4-methylpentanoic acid, 3-hydroxy-4-methoxymandelic acid, 4-hydroxy-3-methoxymendelic acid, 2-hydroxy-2-methylbutanoic acid, 3-(2-hydroxyphenyl) lactic acid, 3-(4-hydroxyphenyl) lactic acid, hexahydromandelic acid, 3-hydroxy-3-methylpentanoic acid, 4-hydroxydecanoic acid, 5-hydroxydecanoic acid and aleuritic acid.

Another type of hydroxyacid suitable for use herein is a hydroxydicarboxylic acid having the following formula:

HOOC(CHOH)ₘ(CH₂)ₙCOOH

wherein m=1,2,3,4,5,6,7,8 or 9; n=3 or an integer up to 23.

The hydrogen attached to the carbon atom pf the (CHOH) group may be substituted by a nonftnctional element such as F, Cl, Br, I, S, or a radical such as a lower saturated or unsaturated alkyl or alkoxy having from I to 9 carbon atoms. Representative hydroxydicarboxylic acids are 2-hydroxypropanedioic acid (tartronic acid), 2-hydroxybutanedioic acid (malic acid), erytliraric acid and threaric acid (tartaric acid), arabiraric acid, ribaric acid, xylaric acid and lyxaric acid, glucaric acid (saccharic acid), galactaric acid (mucic acid), mannaric acid, gularic acid, allaric acid, altraric acid, idaric acid and talaric acid.

Mixtures of hydroxy acids can also be used in the compositions herein. Hydroxy acids are useful herein from the viewpoint of reducing the appearance of wrinkles and improving skin feel and appearance.

Other suitable hydroxy acids for use herein include salicylic acid, retinoic acid, and azelaic acid.

Preferred acidic skin care actives for use herein are salicylic acid and azeleic acid, and mixtures thereof, especially salicylic acid. The salicylic acid is useful herein as a keratolytic active.

The acidic skin care active is generally present at a level of from about 0. 1% to about 10%, preferably from about 0.1% to about 5%, more preferably from about 0.5% to about 3%, by weight of composition.

The acidic skin care active is solubilized in the aqueous phase. The aqueous phase can contain alcohols, for example, solutions based upon C₂-C₆ alcohols, diols and polyols, preferred alcohols being selected from ethanol, dipropylene glycol, butylene glycol, hexylene glycol, and mixtures thereof. Alcohol is preferably present in the compositions herein at a level of from about 1% to about 20%, more preferably at about 8% or less, and especially at about 4% or less. In particularly preferred embodiments the compositions are substantially free of alcohol, that is they comprise about 1% or less by weight of composition.

The composition preferably comprises from about 5% to about 80%, more preferably from about 30% to about 70% by weight of the aqueous phase.

The final skin care active solution preferably has a pH at ambient temperature (25° C.) of less than about pKa+1, where pKa is the logarithmic acidity constant for the fully protonated skin care active. In preferred embodiments, the pH of the final solution is less than about pKa.

The logarithmic acidity constant is thus defined by reference to the equilibrium

H⁺+Hₙ₋₁A=HnA

where HₙA is the fully protonated acid, n is the number of protons in the fully protonated acid and Hₙ₋₁ A is the conjugate base of the acid corresponding to loss of one proton.

The acidity constant for this equilibrium is therefore

$${K}_{n} = \frac{[ {H}_{n} A ]}{[ {H}^{+} ] [ {H}_{n - 1} A ]}$$

and pKₐ=log₁₀Kₙ

For the purposes of this specification, acidity constants are defmed at 25° C. and at zero ionic strength. Literature values are taken where possible (see Stability Constants of Metal-Ion Complexes, Special Publication No. 25, The Chemical Society, London); where doubt arises they are determined by potentiometric titration using a glass electrode. The pKₐ of the acidic skin care active used herein is preferably in the range of from about 1 to about 5.5, more preferably from about 2 to about 4.5, especially from about 2 to about 4.

The pH of the aqueous phase is less than about pH 6, preferably from about pH 2 to about pH 5, more preferably from about pH 2.5 to about pH 4. At pH values of less than about 5 the aqueous phase is preferably free of acid labile species such as polyacrylic or polymethacrylic acids or esters.

The aqueous phase is present in the form of droplets as a discontinuous phase in the water-in-oil emulsions of the present invention.

### Polyoxyethvlene-polyoxvpropvlene ethers of C4 to C22 alcohols

A second essential ingredient in the compositions of the present invention is a nonionic surfactant selected from polyoxyethylene-polyoxypropylene ethers of C4-C22 alcohols, and mixtures thereof. The nonionic surfactant is valuable herein as a solubilising agent for the acidic skin care active in the discontinuous aqueous phase. Suitable polyoxyethylene-polyoxypropylene ethers of C4-C22 alcohols for use herein include those having the general formula:

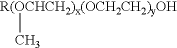

wherein x is in the range of from about 1 to about 35, preferably from about I to about 10, y is in the range of from about 1 to about 45, preferably from about 1 to about 30 and R is a straight chain or branched chain C4 to C22 alkyl group, or a mixture thereof In preferred embodiments (x+y) is greater than or equal to 5, preferably greater than or equal 10, more preferably greater than or equal to 15. The ratio of x:y is in the range from 1:1 to 1 :10.

Examples of suitable R groups in the above formula include cetyl, butyl, stearyl, cetearyl, decyl, lauryl and myristyl.

Examples of suitable polyoxyethylene-polyoxypropylene alcohol ethers include (using CTFA designations) PPG-4-Ceteth-1, PPG-4-Ceteth-5, PPG-4-Ceteth-10, PPG-4-Ceteth-20, PPG-5-Ceteth-20, PPG-8-Ceteth-l,- PPG-8-Ceteth-2, PPG-8-Ceteth-5, PPG-8-Ceteth-10, PPG-8-Ceteth-20, PPG-2-Buteth-3, PPG-2-Buteth-5, PPG-5-Buteth-7, PPG-9-Buteth-12, PPG-28-Buteth-35, PPG-12-Buteth-16, PPG-15-Buteth-20, PPG-20-Buteth-30, PPG-24-Buteth-27, PPG-26-Buteth-26, PPG-33-Buteth-45, PPG-2-Ceteareth-9, PPG-4-Ceteareth-12, PPG-10-Ceteareth-20, PPG-2-Deceth-10, PPG-4-Deceth-4, PPG-6-Deceth-4, PPG-6-Deceth-9, PPG-8-Deceth-6, PPG-2-Isodeceth-4, PPG-2-Isodeceth-6, PPG-2-Isodeceth-9, PPG-2-Isodeceth- 12, PPG-3-Isodeceth-1, PPG-4-Laureth-5, PPG-4-Laureth-2, PPG-4-Laureth-7, PPG-5-Laureth-5, PPG-25-Laureth-25, PPG-3-Myreth-11, PPG-3-Myreth-3 and PPG-9-Steareth-3.

Preferred polyoxyethylene-polyoxypropylene ethers for use herein are ethers of C8 to C16 alcohols having the formula (I) wherein x is from 2 to 12 and y is from 10 to 30 and where the ratio of x:y is in the range of from about 1:2 to about 1:8.

Particularly preferred polyoxyethylene-polyoxypropylene ethers of C4 to C22 alcohols for use herein are those having the formula (I) above wherein R is cetyl and wherein x is in the range of from about 4 to about 8, and wherein y is in the range of from about 15 to about 25, and the ratio of x:y is in the range of from about 1:3 to about 1:5. A particularly preferred ether from the viewpoint of improving solubilisation of the acidic skin care active is PPG-5-Ceteth-20, which is available under the tradename Procetyl AWS.

### Oil Phase

The composition of the present invention is in the form of a water-in-oil emulsion. The oil phase can comprise silicone oils, non-silicone organic oils, or mixtures thereof.

In preferred embodiments the oil phase comprises a mixture of volatile silicones and non-volatile silicones. The silicones are useful herein for providing skin conditioning properties. The silicone fluid is present in an amount of from about 1% to about 50% by weight. Suitable volatile silicones include cyclic and linear volatile polyorganosiloxanes. The term “nonvolatile” as used herein shall mean the material has a vapour pressure of no more than 0.1 mm Hg at one atmosphere and 25° C. The term “volatile” as used herein shall mean materials which are not nonvolatile or which have a vapour pressure at the same conditions of more than 0.1mm Hg.

A description of various volatile silicones is found in Todd, et al.. “Volatile Silicone Fluids for Cosmetics”, 91 Cosmetics and Toiletries 27-32 (1976).

Preferred cyclic silicones include cyclic dimethyl siloxane chains containing an average of from about 3 to about 9 silicon atoms, preferably from about 4 to about 5 silicon atoms. Preferred linear silicones include the polydimethylsiloxanes containing an average of from about 3 to about 9 silicon atoms. The linear volatile silicones generally have viscosities of less than about 5 centistokes at 25° C., while the cyclic materials have viscosities of less than about 10 centistokes. Examples of silicone oils useful in the present invention include: Dow Corning 344, Dow Coming 21330, Dow Coming 345, and Dow Coming 200 (manufactured by the Dow Corning Corporation): Silicone 7207 and Silicone 7158 (manufactured by the Union Carbide Corporation). SF:202 (manufactured by General Electric) and SWS-03314 (manufactured by Stauffer Chemical).

The nonvolatile silicones will have vapour pressures as previously defined, and preferably will have an average viscosity of from about 10 to about 100,000 cps at 25° C, more preferably from about 100 to about 10,000 cps, even more preferably from about 500 to about 6000 cps. Lower viscosity non-volatile silicone conditioning agents, however, can also be used. Viscosity can be measured by means of a glass capillary viscometer as set forth in Dow Corning Corporate Test Method CTMOO04, Jul. 20, 1970.

Suitable non-volatile silicone fluids for use herein include polyalkyl siloxanes, polyaryl siloxanes, polyalkylaryl siloxanes, polysiloxanes with amino functional substitutions, polyether siloxane copolymers, and mixtures thereof. The siloxanes useful in the present invention may be substituted and/or endcapped with any number of moieties, so long as the material remains suitable for use in a topical cosmetic product, including, for example, methyl, hydroxyl, ethylene oxide, propylene oxide, amino and carboxyl. However, other silicone fluids having skin conditioning properties may be used. The non-volatile polyalkyl siloxane fluids that may be used include, for example, polydimethylsiloxanes. These siloxanes are available, for example, from the General Electric Company as a Viscasil (RTM) series and from Dow Corning as the Dow Corning 200 series. Preferably, the viscosity ranges from about 10 mm².s⁻¹ to about 100,000 mm².s^{<1} at 25° C. The polyalkylaryl siloxane fluids that may be used, also include, for example, polymethylphenylsiloxanes. These siloxanes are available, for example, from the General Electric Company as SF 1075 methyl phenyl fluid or from Dow Coming as 556 Cosmetic Grade Fluid. The polyether siloxane copolymer that may be used includes, for example, a polypropylene oxide modified dimethylpolysiloxane (e.g., Dow Coming DC-1248) although ethylene oxide or mixtures of ethylene oxide and propylene oxide may also be used.

References disclosing suitable silicone fluids include U.S. Pat. No. A-2,826,551, Green; U.S. Pat. No. A-3,964,500, Drakoff, issued Jun. 22, 1976; U.S. Pat. No. A-4,364,837, Pader; and GB-A-849,433, Woolston. In addition, Silicone Compounds distributed by Petrarch Systems Inc., 1984 provides an extensive (though not exclusive) listing of suitable silicone fluids.

Preferred non-volatile silicones for use herein include polydiorganosiloxane-polyoxyalkylene copolymers containing at least one polydiorganosiloxane segment and at least one polyoxyalkylene segment. The polydiorganosiloxane segment has the general formula:

R_{b}SiO_{(4-b)/2}

siloxane units wherein b has a value of from about 0 to about 3, inclusive, there being an average value of approximately two R radicals per silicon for all siloxane units in the copolymer, and R denotes a radical selected from methyl, ethyl, vinyl, phenyl and a divalent radical bonding said polyoxyalkylene segment to the polydiorganosiloxane segment. The polyoxyalkylene segment has an average molecular weight of at least about 500, preferably at least about 1000, and comprising from about 0 to about 50 mol percent polyoxypropylene units and from about 50 to about 100 mol percent polyoxyethylene units, at least one terminal portion of said polyoxyalkylene segment being grafted to, or covalently bonded directly or indirectly to a polydiorganosiloxane segment, any terminal portion of said polyoxyalkylene segment not bonded to said polydiorganosiloxane segment being satisfied by a terminating radical; the weight ratio of polydiorganosiloxane segments to polyoxyalkylene segments in said copolymer preferably having a value of from about 2 to about 8. Such polymers are described in U.S. Pat. No. A-4,268,499. Preferred for use herein are polydiorganosiloxane-polyoxyalkylene copolymers having the general formula:

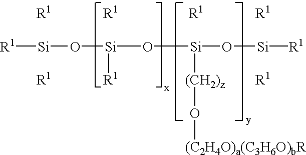

wherein R¹ is selected from C1 to C5 alkyl groups, preferably methyl, z is in the range of from 1 to 4, x and y are selected such that the weight ratio of polydiorganosiloxane segments to polyoxalkylene segments is from about 2 to about 8, the mol ratio of a:(a+b) is from about 0.5 to about 1, and R is a chain terminating group, especially selected from hydrogen; hydroxyl; alkyl, such as methyl, ethyl, propyl, butyl, benzyl; aryl, such as phenyl; alkoxy such as methoxy, ethoxy, propoxy, butoxy; benzyloxy; aryloxy, such as phenoxy; alkenyloxy, such as vinyloxy and allyloxy; acyloxy, such as acetoxy, acryloxy and propionoxy and amino, such as dimethylamino.

More preferred for use herein are polydiorganosiloxane-polyoxyalkylene copolymers having the formula:

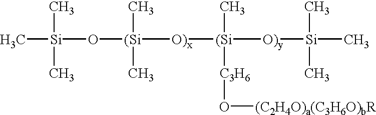

wherein x, y and R are as defined above.

The number of and average molecular weights of the segments in the copolymer are such that the weight ratio of polydiorganosiloxane segments to polyoxyalkylene segments in the copolymer is preferably from about 2.5 to about 4.0.

Suitable copolymers are available commercially under the tradenames Belsil (RTM) from Wacker-Chemie GmbH, Geschaftsbereich S, Postfach D-8000 Munich 22 and Abil (RTM) from Th. Goldschmidt Ltd,. Tego House, Victoria Road, Ruislip, Middlesex, HA4 OYL. Particularly preferred for use herein are Belsil (RTM) 6031, Abil (RTM) B88183, DC3225C, DC5200, Abil We09 and Abil EM90. A preferred silicone herein is known by its CTFA designation as dimethicone copolyol.

The compositions of the present invention preferably comprises from about 1% to about 50% by weight of composition of silicone oil phase. The silicone oil phase preferably comprises from about 0.01% to about 25%, more preferably from about 0.05% to about 10% by weight of the silicone oil phase of non-volatile silicones. The silicone oil phase preferably comprises from about 75% to about 99.99%, more preferably from about 90% to about 99.95% by weight of volatile silicones.

The oil phase in the water-in-oil emulsions of the present invention can also comprise one or more non-silicone organic oil, such as natural or synthetic oil selected from mineral, vegetable, and animal oils, fats and waxes, fatty acid esters, fatty alcohols, fatty acids and mixtures thereof, which ingredients are useful for achieving emollient cosmetic properties. It will be understood that the oil phase may contain, for example, up to about 25%, preferably up to only about 10% of oil phase soluble emulsifier ingredients. Such ingredients are not to be considered as oil phase components from the viewpoint of determining the oil phase level.

Suitable organic oils for use herein include, for example, optionally hydroxy-substituted C₈-C₅₀ unsaturated fatty acids and esters thereof, C₁-C₂₄ esters of C₈-C₃₀ saturated fatty acids such as isopropyl myristate, isopropyl palmitate, cetyl palmitate and octyldodecylmyristate (Wickenol 142), beeswax, saturated and unsaturated fatty alcohols such as behenyl alcohol and cetyl alcohol, hydrocarbons such as mineral oils, petrolatum and squalane, fatty sorbitan esters (see U.S. Pat. No. 3,988,255, Seiden, issued Oct. 26, 1976), lanolin and lanolin derivatives, animal and vegetable triglycerides such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grapeseed oil, shea butter, shorea butter, and sunflower seed oil and C₁ C₂₄ esters of dimer and trimer acids such as diisopropyl dimerate, diisostearylmalate, diisostearyldimerate and triisostearyltrimerate. Of the above, highly preferred are the mineral oils, petrolatums, unsaturated fatty acids and esters thereof and mixtures thereof.

### Optional Ingredients

The compositions of the present invention can further comprise solubilizing agents in addition to the polyoxyethylene-polyoxypropylene alcohol ether. Suitable additional solubilizing agents for use herein are selected from pyrrolidone-based solubilising agents, polyethylene glycol based nonionic surfactants having an HLB of greater than about 15, preferably greater than about 18, and mixtures thereof. The additional solubilizing agent herein is preferably present at a level of from about 0.1% to about 10%, more preferably from about 0.1% to about 5%, especially from about 0.5% to about 2% by weight of composition.

Pyrrolidone-based solubilising agents suitable for use herein include polyvinylpyrrolidone or C₁-C₄ alkyl polyvinylpyrrolidone having a molecular weight (viscosity average) in the range from about 1500 to about 1,500,000, preferably from about 3000 to about 700,000, more preferably from about 5000 to about 100,000. Suitable examples of pyrrolidone-based solubilising agents are polyvinylpyrrolidone (PVP) (or povidone) and butylated polyvinylpyrrolidone. The most preferred pyrrolidone-based solubilising agent herein is polyvinylpyrrolidone. PVP is commercially available under the trade name Luviskol (RTM) from BASF. A preferred PVP solubilising agent herein is Luviskol K17 which has a viscosity-average molecular weight of about 9,000. Other pyrrolidone-based solubilising agents for use herein include C₁-C₁₈ alkyl or hydroxyalkyl pyrrolidones such as lauryl pyrrolidone.

The pyrrolidone-based solubilising agent is preferably present in the composition herein in a level of from about 0.1% to about 10%, more preferably from about 0.1% to about 5%, especially from about 0.5% to about 2% by weight of composition. The weight ratio of acidic skin care active: pyrrolidone-based solubilising agent is preferably in the range from about 10:1 to about 1:10, more preferably from about 5:1 to about 1:5.

Preferred embodiments of the invention additionally comprise from about 0.01% to about 5% by weight of an additional acid or salt thereof which is soluble in water at pH values of less than or equal to the pKa of the corresponding acid, for example, an acid selected from citric acid, boric acid, and salts, and mixtures thereof. These materials are valuable herein in combination with the pyrrolidone-based complexing agent from the viewpoint of aiding solubilization of the acidic skin care active. Particularly preferred herein from this viewpoint is a sodium salt of citric acid. In preferred embodiments, the acid or salt thereof is soluble to a level of at least 5% w/w at 25° C.

Preferred embodiments herein comprise a pigment or mixture of pigments. It is a feature of the composition herein that the pigment and anti-acne active have excellent overall compatability and colour stability. Suitable pigments for use herein can be inorganic and/or organic. Also included within the term pigment are materials having a low colour or lustre such as matte finishing agents, and also light scattering agents. Examples of suitable pigments are iron oxides, rutile titanium dioxide, anatase titanium dioxide, ferric oxide, ferrous oxide, chromium oxide, chromium hydroxide, manganese violet, acylglutamate iron oxides, ultramarine blue, D&C dyes, carmine, and mixtures thereof. Depending upon the type of make-up composition, eg. foundation or blusher, a mixture of pigments will normally be used.

The foundation composition can also include at least one matte finishing agent. The finction of the matte finishing agent is to hide skin defects and reduce shine. Such cosmetically acceptable inorganic agents, i.e., those included in the CTFA Cosmetic Ingredient Dictionary, Third Ed., as silica, hydrated silica, silicone-treated silica beads, mica, talc, polyethylene, titanium dioxide, bentonite, hectorite, kaolin, chalk, diatomaceous earth, attapulgite zinc oxide and the like may be utilized. Of particular usefulness as a matte finishing agent is low lustre pigment such as titanated mica (mica coated with titanium dioxide) coated with barium sulfate. Of the inorganic components useful as a matte finishing agent low lustre pigment, talc, polyethylene, hydrated silica, kaolin, titanium dioxide and mixtures thereof are particularly preferred. Materials suitable for use herein as light-scattering agents can be generally described as spherical shaped inorganic materials having a particle size of up to about 100 microns, preferably from about 5 to about 50 microns, for example spherical silica particles.

Other examples of pigments include lakes of organic colorants such as FD&C Red No. 7 calcium lake, FD&C Yellow No. 5 aluminium lake, D&C Red No. 9 barium lake, and D&C Red No. 30.

The preferred pigments for use herein from the viewpoint of moisturisation, skin feel, skin appearance and emulsion compatibility are treated pigments. The pigments can be treated with compounds such as amino acids such as lysine, silicones, lauroyl, collagen, polyethylene, lecithin and ester oils. The more preferred pigments are the silicone (polysiloxane) treated pigments.

A highly preferred pigment for use herein is a pigment which has been coated with organosilicon component selected from a polyorganosiloxane or a silane wherein the coated pigment has a hydrogen potential of less than about 2.0, preferably less than about 1.0, more preferably less than about 0.5 ml, and especially less than about 0.1 ml H₂/ g of coated pigment. The pigment preferred for use herein is in particulate form. The pigment is incorporated into the continuous oil phase in the compositions herein. The coatings used can be bonded to the pigment surface by covalent bonding, physical adsorption or adhesion, preferably by covalent bonding to the surface of the pigment. The function of the coatings herein is to hydrophobically-modify the pigments so that thay are “wettable” in the continuous silicone phase of the water-in-silicone emulsions. The coated pigment is also useful herein from the viewpoint of reducing hydrogen gas evolution and improving product stability.

Without wishing to be limited by theory it is believed that although the pigments are present in the oil phase of the water-in-oil emulsion, hydrogen ions from the aqueous phase can pass through the interface of the emulsion into the oil phase, where they are available to react with the pigment coatings, e.g. to give off hydrogen gas. However, by using organosilicon-coated pigments having a hydrogen potential of less than about 2 ml H₂/g of coated pigment, hydrogen gas generation is reduced. The hydrogen potential of the coated pigment is measured herein using the following test method:

A dispersion of the coated pigment containing 20g of coated pigment is placed in a flask on a magnetic stirrer and 100ml of a 2% ethanolic solution of potassium hydroxide is added with stirring at ambient temperature. The hydrogen gas which is evolved is collected in a second flask at ambient temperature and pressure (25° C, 1 At). The hydrogen gas released can therefore be volumetrically measured.

A wide variety of organosilicon components can be used for treating the pigments herein. A suitable polyorganosiloxane herein is selected from:

(A) material of the formula:

(R¹ )3 SiO-(Si(R²R³)0)p-Si(R²R³)OA₂

wherein p is 1 to 1000, preferably from 1 to 100, A₂ is hydrogen or an alkyl group having from 1 to 30 carbon atoms, R¹ is a C₁-C₃₀ alkyl, preferably methyl, R² and R³ are independently selected from a C₁-C₃₀ alkyl and a phenyl, preferably wherein R² and R³ are both methyl or wherein R² is methyl and R³ is phenyl; or

(B) material of the formula:

(R¹ )₃SiO(Si(R²)(H)-O)ᵢ-Si(R¹)₃

wherein i is 1 to 1000, preferably 1 to 100, and wherein R¹ and R² are as defined above for formula (A).

In preferred embodiments the organosilicon component is selected from a silane. The silane can be selected from material of the formula:

(C) A₁SiX₁X₂X₃

wherein A is an alkyl or alkenyl group having from 1 to 30 carbon atoms, and XI, X₂ and X₃ are independently C₁-C₄ alkoxy preferably methoxy or ethoxy, or halo, preferably chloro.

When the pigment herein is treated with silane material having the formula (C) described herein above a pigment having the following formula (1) is produced:

P-0-Si(OH)(A)-[-O-Si(OH)(A)-]₀-₁₀₀-OH

wherein P is an atom in the pigment surface and each A is an alkyl or alkenyl group having up to 30 carbon atoms. A number of adjacent polysiloxane chains as shown in formula (1) can be cross-linked through oxygen atoms to form a polysiloxane chain with up to 100 repeating

-Si(-OP)-O-units that extend along the pigment surface, in addition to the polysiloxane chain which extends away from the pigment surfact. Examples of linear or branched alkyl groups are methyl, ethyl, propyl, butyl, pent l,, hexyl, heptyl, octyl, and so forth up to octadecyl. . “Alkenyl” includes carbon chains with one or more double bonds; examples of such groups include ethylene, propylene, acrylyl, methacrylyl, and residues of unsaturated fatty acids such as oleic (C_{1 7}C₃₃-), linoleic (C₁₇H₃₁-), and linolenic (C₁₇H₂₉-).

When the pigment herein is treated with polyorganosiloxane material having the formula (A) described hereinabove a pigment having the following formula (2) is produced:

P-O-(Si(R²R³)0)p-Si(R¹)₃ (2)

wherein p is 1-1000, preferably 1 to 100, R¹, R² and R³ are as defined above for formula (A) and P is an atom in the pigment surface.

When the pigment herein is treated with polyorganosiloxane material having the formula (B) described hereinabove a pigment having the follo wing formula (3) is produced: (R¹)₃SiO-[Si(R²)(-OP)-O-]p-Si(R¹)₃ (3)

wherein each P is an atom in the pigment surface, p is from I to 1000, preferably from 1 to 100, R¹ and R² are as defined above in formula (B) uid in which each of the up to 100 repeating (Si-0) units is bonded through an oxygen atom to the pigment surface.

The pigment (or a mixture of two or more pigments) can be coated by placing it in dry, finely divided form in a mixer, adding the organosilicon component, and mixing. The organosilicon coating is preferably present at a level of from about 0.01% to about 5%, more preferably from about 0.1% to about 4%, and especially from about 0.5% to about 2%, by weight of the organosilicon coated pigment.

The most preferred coated pigment from the viewpoint of reducing hydrogen gas evolution and improving product stability is Cardre 70429.

The total concentration of the coated pigment may be from about 0.1 to about 25% by weight and is preferably from about 1 to about 15%, more preferably from about 8% to about 12% by weight of the total composition, the exact concentration being dependent to some extent upon the specific mixture of pigments selected for use in a foundation make-up or blusher to achieve the desired shades. The preferred compositions contain from about 2% to about 20% by weight of titanium dioxide and most preferably from about 5% to about 10% by weight of titanium dioxide.

A highly preferred component of the compositions herein is a humectant or mixture of humectants. The humectant or mixture of humectants herein is present in an amount of from about 0.1% to about 30% preferably from about 1% to about 25%, and more preferably from about 1% to about 10% by weight of composition. Suitable humectants are selected from glycerine and polyglycerylmethacrylate lubricant having a viscosity at 25° C. of 300,000 to 1,100,000 cps; a specific gravity at 25° C. of 1 to 1.2g/ml, a pH of 5.0 to 5.5; a bound water content of 33 to 58%; and, a free water content from 5 to 20%.

The humectant can be incorporated at least partly into the oil phase of the water-in-oil emulsion. The oil phase preferably comprises from about 0.1% to about 10%, more preferably from about 0.1% to about 3% by weight of humectant on a composition basis. The humectant can be introduced into the oil phase in the form of a mixture with or incorporated within a particulate lipophilic or hydrophobic carrier material.

Polyglycerylmethacrylate lubricants having the desired properties are marketed by Guardian Chemical Corporation under the trademark “Lubrajel”. The “Lubrajels” identified as “Lubrajel DV”, “Lubrajel MS”, and “Lubrajel CG” are preferred in the present invention. The gelling agents sold under these trademarks contain about 1% propylene glycol.

Other suitable humectants include sorbitol, panthenols, propylene glycol, dipropylene glycol, butylene glycol, hexylene glycol, alkoxylated glucose derivatives, such as Glucam (RTM) E-20, hexanetriol, and glucose ethers, and mixtures thereof.

The panthenol moisturiser can be selected from D-panthenol ([R]-2,4-dihydroxy-N-[3-hydroxypropyl)]-3,3-dimethylbutamide), DL-panthenol, calcium pantothenate, royal jelly, panthetine, pantotheine, panthenyl ethyl ether, pangamic acid, pyridoxin, pantoyl lactose and Vitamin B complex.

The preferred humectant herein is glycerine. Chemically, glycerine is 1,2,3-propanetriol and is a product of commerce.

A preferred component of the compositions herein, in addition to the organic amphiphilic surfactant is a polyol ester skin conditioning agent. The compositions of the present invention preferably comprise from about 0.01% to about 20%, more preferably from about 0.1% to about 15%, and especially from about 1% to about 10% by weight of the polyol ester. The level of polyol ester by weight of the oil in the composition is preferably from about 1% to about 30%, more preferably from about 5% to about 20%.

The polyol ester preferred for use herein is a nonocclusive liquid or liquifiable polyol carboxylic acid ester. These polyol esters are derived from a polyol radical or moiety and one or more carboxylic acid radicals or moieties. In other words, these esters contain a moiety derived from a polyol and one or more moieties derived from a carboxylic acid. These carboxylic acid esters can also be derived from a carboxylic acid. These carboxylic acid esters can also be described as liquid polyol fatty acid esters, because the terms carboxylic acid and fatty acid are often used interchangeably by those skilled in the art.

The preferred liquid polyol polyesters employed in this invention comprise certain polyols, especially sugars or sugar alcohols, esterified with at least four fatty acid groups. Accordingly, the polyol starting material must have at least four esterifiable hydroxyl groups. Examples of preferred polyols are sugars, including monosaccharaides and disaccharides, and sugar alcohols. Examples of monosaccharides containing four hydroxyl groups are xylose and arabinose and the sugar alcohol derived from xylose, which has five hydroxyl groups, i.e., xylitol. The monosaccharide, erythrose, is not suitable in the practice of this invention since it only contains three hydroxyl groups, but the sugar alcohol derived from erythrose, i.e., erythritol, contains four hydroxyl groups and accordingly can be used. Suitable five hydroxyl group-containing monosaccharides are galactose, fructose, and sorbose. Sugar alcohols containing six -OH groups derived from the hydrolysis products of sucrose, as well as glucose and sorbose, e.g., sorbitol, are also suitable. Examples of disaccharide polyols which can be used include maltose, lactose, and sucrose, all of which contain eight hydroxyl groups.

Preferred polyols for preparing the polyesters for use in the present invention are selected from the group consisting of erythritol, xylitol, sorbitol, glucose, and sucrose. Sucrose is especially preferred.

The polyol starting material having at least four hydroxyl groups is esterified on at least four of the -OH groups with a fatty acid containing from about 8 to about 22 carbon atoms. Examples of such fatty acids include caprylic, capric, lauric, myristic, myristoleic, palmitic, palmitoleic, stearic, oleic, ricinoleic, linoleic, linolenic, eleostearic, arachidic, arachidonic, behenic, and erucic acid. The fatty acids can be derived from naturally occurring or synthetic fatty acids; they can be saturated or unsaturated, including positional and geometrical isomers. However, in order to provide liquid polyesters preferred for use herein, at least about 50% by weight of the fatty acid incorporated into the polyester molecule should be unsaturated. Oleic and linoleic acids, and mixtures thereof, are especially preferred.

The polyol fatty acid polyesters useful in this invention should contain at least four fatty acid ester groups. It is not necessary that all of the hydroxyl groups of the polyol be esterified with fatty acid, but it is preferable that the polyester contain no more than two unesterified hydroxyl groups. Most preferably, substantially all of the hydroxyl groups of the polyol are esterified with fatty acid, i.e., the polyol moiety is substantially completely esterified. The fatty acids esterified to the polyol molecule can be the same or mixed, but as noted above, a substantial amount of the unsaturated acid ester groups must be present to provide liquidity.

To illustrate the above points, a sucrose fatty triester would not be suitable for use herein because it does not contain the required four fatty acid ester groups. A sucrose tetra-fatty acid ester would be suitable, but is not preferred because it has more than two unesterified hydroxyl groups. A sucrose hexa-fatty acid ester would be preferred because it has no more than two unesterified hydroxyl groups. Highly preferred compounds in which all the hydroxyl groups are esterified with fatty acids include the liquid sucrose octa-substituted fatty acid esters.

The following are non-limiting examples of specific polyol fatty acid polyesters containing at least four fatty acid ester groups suitable for use in the present invention: glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof.

As noted above, highly preferred polyol fatty acid esters are those wherein the fatty acids contain from about 14 to about 18 carbon atoms. The preferred liquid polyol polyesters preferred for use herein have complete melting points below about 30° C., preferably below about 27.5° C., more preferably below about 25° C. Complete melting points reported herein are measured by Differential Scanning Calorimetry (DSC).

The polyol fatty acid polyesters suitable for use herein can be prepared by a variety of methods well known to those skilled in the art. These methods include: transesterification of the polyol with methyl, ethyl or glycerol fatty acid esters using a variety of catalysts; acylation of the polyol with a fatty acid chloride; acylation of the polyol with a fatty acid anhydride; and acylation of the polyol with a fatty acid, per se. See U.S. Pat. No. 2,831,854; U.S. Pat. No. 4,005,196, to Jandacek, issued Jan. 25, 1977; U.S. Pat. No. 4,005,196, to Jandacek, issued Jan. 25, 1977.

The make-up compositions of the present invention can also comprise a particulate cross-linked hydrophobic acrylate or methacrylate copolymer. This copolymer is particularly valuable for reducing shine and controlling oil while helping to provide effective moisturization benefits. The cross-linked hydrophobic polymer is preferably in the form of a copolymer lattice with at least one active ingredient dispersed uniformly throughout and entrapped within the copolymer lattice. Alternatively, the hydrophobic polymer can take the form of a porous particle having a surface area (N₂-BET) in the range from about 50 to 500, preferably 100 to 300m²/g and having the active ingredient absorbed therein.

The cross-linked hydrophobic polymer when used herein is in an amount of from about 0.1% to about 10% by weight and is preferably incorporated in the external silicone-containing oil phase. The active ingredient can be one or more or a mixture of skin compatible oils, skin compatible humectants, emollients, moisturizing agents and sunscreens. The polymer material is in the form of a powder, the powder being a combined system of particles. The system of powder particles forms a lattice which includes unit particles of less than about one micron in average diameter, agglomerates of fused unit particles of sizes in the range of about 20 to 100 microns in average diameter and aggregates of clusters of fused agglomerates of sizes in the range of about 200 to 1,200 microns in average diameter.

The powder material of the present invention which can be employed as the carrier for the active ingredient can be broadly described as a cross-linked “post absorbed” hydrophobic polymer lattice. The powder preferably has entrapped and dispersed therein, an active which may be in the form of a solid, liquid or gas. The lattice is in particulate form and constitutes free flowing discrete solid particles when loaded with the active material. The lattice may contain a predetermined quantity of the active material. The polymer has the structural formula:

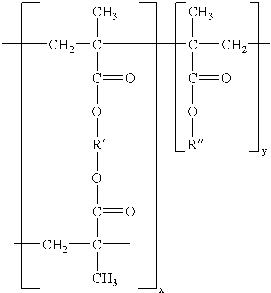

where the ratio of x to y is 80:20, R′ is -CH₂CH₂- and R¹¹ is -(CH₂)_{1 1}CH₃.

The hydrophobic polymer is a highly crosslinked polymer, more particularly a highly cross-linked polymethacrylate copolymer. The material is manufactured by the Dow Coming Corporation, Midland. Michigan, USA, and sold under the trademark POLYTRAP (RTM) . It is an ultralight free-flowing white powder and the particles are capable of absorbing high levels of lipophilic liquids and some hydrophilic liquids while at the same time maintaining a free-flowing powder character. The powder structure consists of a lattice of unit particles less than one micron that are fuised into agglomerates of 20 to 100 microns and the agglomerates are loosely clustered into macro-particles or aggregates of about 200 to about 1200 micron size. The polymer powder is capable of containing as much as four times its weight of fluids, emulsions, dispersions or melted solids.

Adsorption of actives onto the polymer powder can be accomplished using a stainless steel mixing bowl and a spoon, wherein the aci ve is added to the powder and the spoon is used to gently fold the active into tile polymer powder. Low viscosity fluids may be adsorbed by addition of the fluids to a sealable vessel containing the polymer and then tumbling the mater als until a consistency is achieved. More elaborate blending equipment such as ribbon or twin cone blenders can also be employed. The preferred active ingredient for use herein is glycerine. Preferably, the weight ratio of humectant: carrier is from about 1:4 to about 3:1.

Also suitable as a highly cross-linked polymethacrylate copolymer is Microsponges 5647. This takes the form of generally spherical particles of cross-linked hydrophobic polymer having a pore size of from about 0.01 to about 0.05pm and a surface area of 200-300m²/g. Again, it is preferably 1laded with humectant in the levels described above.

The compositions of the invention can also contain a -nydrophilic gelling agent at a level preferably from about 0.01% to about 10%, more preferably from about 0.02% to about 2%, and especially from about 0.02% to al out 0.5%. The gelling agent preferably has a viscosity (1% aqueous solution, 200(, Brookfield RVT) of at least about 4000 mPa.s, more preferably at least about 1(,000 mPa.s and especially at least 50,000 mpa.s.

Suitable hydrophilic gelling agents can generally be described atwater-soluble or colloidally water-soluble polymers, and include cellulose ethers (e.g. hydroxyethyl cellulose, methyl cellulose, hydroxypropylmethyl cellulose), polyvinylalcohol, polyquatemium- 10, guar gum, hydroxypropyl guar gum and xanthan gum. Among suitable hydrophilic gelling agents are acrylic acid/alkyl acrylate copolymers and the carboxyvinyl polymers sold by the B.F. Goodrich Company under the trade mark of Carbopol resins. These resins consist essentially of a colloidally water-soluble polyalkenyl polyether crosslinked polymer of acrylic acid crosslinked with from 0.75% to 2.00% of a crosslinking agent such as for example polyallyl sucrose or polyallyl pentaerythritol. Examples include Carbopol 934, Carbopol 940, Carbopol 950, Carbopol 980, Carbopol 951 and Carbopol 981. Carbopol 934 is a water-soluble polymer of acrylic acid crosslinked with about 1% of a polyallyl ether of sucrose having an average of about 5.8 allyl groups for each sucrose molecule. Also suitable for use herein are hydrophobically-modified cross-linked polymers of acrylic acid having amphipathic properties available under the Trade Name Carbopol 1382, Carbopol 1342 and Pemulen TR-1 (CTFA Designation: Acrylates/10-30 Alkyl Acrylate Crosspolymer). A combination of the polyalkenyl polyether cross-linked acrylic acid polymer and the hydrophobically modified cross-linked acrylic acid polymer is also suitable for use herein. Other suitable gelling agents suitable for use herein are oleogels such as trihydroxystearin and aluminium magnesium hydroxy stearate. The gelling agents herein are particularly valuable for providing excellent stability characteristics over both normal and elevated temperatures.

Neutralizing agents suitable for use in neutralizing acidic group containing hydrophilic gelling agents herein include sodium hydroxide, potassium hydroxide, ammonium hydroxide, monoethanolamine, diethanolamine and triethanolamine.

The make-up compositions herein can additionally comprise an emollient. Emollients suitable for the compositions of the present invention include natural and synthetic oils selected from mineral, vegetable, and animal oils, fats and waxes, fatty acid esters, fatty alcohols, alkylene glycol and polyalkylene glycol ethers and esters, fatty acids and mixtures thereof.

Suitable emollients for use herein include, for example, optionally hydroxy-substituted C₈-C₅₀ unsaturated fatty acids and esters thereof, C₁-C₂₄ esters of C₈-C₃₀ saturated fatty acids such as isopropyl myristate, cetyl palmitate and octyldodecylmyristate (Wickenol 142), beeswax, saturated and unsaturated fatty alcohols such as behenyl alcohol and cetyl alcohol, hydrocarbons such as mineral oils, petrolatum and squalane, fatty sorbitan esters (see U.S. Pat. No. A-3988255, Seiden, issued Oct. 26, 1976), lanolin and lanolin derivatives, such as lanolin alcohol ethoxylated, hydroxylated and acetylated lanolins, cholesterol and derivatives thereof, animal and vegetable triglycerides such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grapeseed oil, and sunflower seed oil and C₁ -C₂₄ esters of dimer and trimer acids such as diisopropyl dimerate, diisostearylmalate, diisostearyldimerate and triisostearyltrimerate.

Preferred emollients are selected from hydrocarbons such as isohexadecane, mineral oils, petrolatum and squalane, lanolin alcohol, and stearyl alcohol. These emollients may be used independently or in mixtures and may be present in the composition of the present invention in an amount from about 1% to about 30% by weight, and preferably are present in an amount from about 5% to about 15% by weight of the total composition.

The composition may also contain additional materials such as, for example, fragrances, sun-screens, preservatives, electrolytes such as sodium chloride, proteins, antioxidants, chelating agents and water-in-oil emulsifiers as appropriate. Another optional component of the make-up composition is one or more ultraviolet absorbing agents. Ultraviolet absorbing agents, often described as sunscreening agents, can be present in a concentration in the range of between about 1% and about 12% by weight, based on the total weight of composition. Preferably, the UV absorbing agents constitute between about 2% and 8% by weight. More preferably, the UV absorbing agents can be present in the composition in a concentration range of between about 4% and about 6% by weight. Of the ultraviolet absorbing agents suitable for use herein, benzophenone-3, octyl dimethyl PABA (Padimate 0), Parsol MCX, and mixtures thereof are particularly preferred.

Another optional but preferred component herein is one or more additional chelating agents, preferably in the range of from about 0.02% to about 0. 10% by weight, based on the total weight of the composition. Preferably, the chelating agent is present in a concentration in the range of between about 0.03% and about 0.07% by weight, based on the total weight of the composition. Among the chelating agents that may be included in the composition is tetrasodium EDTA.

Another optional but preferred component of the foundation composition is one or more preservatives. The preservative concentration in the foundation composition, based on the total weight of that composition, is in the range of between about 0.05% and about 0.8% by weight, preferably between about 0.1% and about 0.3% by weight. Suitable preservatives for use herein include sodium benzoate and propyl paraben, and mixtures thereof.

The balance of the composition of the present invention is deionized water. The composition preferably comprises from about 20% to about 95%, more preferably from about 30% to about 70% by weight of the oil phase, and from about 5% to about 80%, more preferably from about 30% to about 70% by weight of the water phase.

The cosmetic compositions of the present invention can be in the form of foundations, blushers, concealers, compact powders, and the like, preferably as foundations and concealers.

The table below shows examples of cosmetic compositions of the present invention.

| | I/% | II/% | III/% | IV/% | V/% | VI/% |
|---|---|---|---|---|---|---|
| A | | | | | | |
| Cyclomethicone [DC 21330]¹ | 15.2 | 16.0 | 14.8 | 15.3 | 15.7 | 18.8 |
| Cyclomethicone/Dimethicone | 15.0 | 10.0 | 18.5 | 15.2 | 17.25 | 8.0 |
| copolyol [90:10] [DC3225C]¹ | | | | | | |
| SEFA Cottonate² | 2.0 | 4.0 | 0.0 | 0.0 | 0.0 | 0.0 |
| Microsponge¹ | 3.0 | 0.5 | 0.75 | 0.75 | 1.0 | 0.0 |
| B | | | | | | |
| Mica | 0.1 | 0.15 | 0.1 | 0.12 | 0.1 | 0.0 |
| Titanium Dioxide | 8.25 | 6.0 | 14.2 | 7.85 | 8.5 | 8.5 |
| (Cardre 70429)³ | | | | | | |
| Zinc Oxide | 0.4 | 6.0 | 0 | 2.0 | 0 | 4.0 |
| C | | | | | | |
| DryFlow¹ | 0.0 | 2.5 | 0 | 2.0 | 1.5 | 3.5 |
| D | | | | | | |
| Yellow iron oxide | 0.55 | 0.6 | 0.49 | 2.0 | 0.58 | 2.1 |
| Red iron oxide | 0.3 | 2.0 | 0.28 | 0.24 | 0.4 | 0.9 |
| Black iron oxide | 0.1 | 0.08 | 0.05 | 0.12 | 0.15 | 0.6 |
| E | | | | | | |
| Durachem⁸ | 0.1 | 0.1 | 0.0 | 0.1 | 0.1 | 0.0 |
| Waxenol⁹ | 0.3 | 0.0 | 0.3 | 0.3 | 0.3 | 0.0 |
| F | | | | | | |
| Cyclomethicone | 1 | 1.5 | 1 | 1.2 | 1 | 0.0 |
| [DC 21330]¹ | | | | | | |
| Thixin R⁴ | 0.3 | 0.3 | 0.3 | 0.3 | 0.3 | 0.0 |
| Propyl paraben | 0.0 | 0.2 | 0.1 | 0.15 | 0.0 | 0.0 |
| G | | | | | | |
| Ethylene Brassylate | 0.1 | 0.0 | 0.1 | 0.0 | 0.1 | 0.1 |
| Rose Water Concentrate | 0.0 | 0.0 | 0.0 | 0.0 | 0.0 | 0.001 |
| H | | | | | | |
| Ethanol | 4 | 0 | 2 | 0 | 5 | 4 |
| Polyvinyl- | 1 | 1.8 | 0.8 | 1.5 | 1.2 | 1 |
| pyrrolidone | | | | | | |
| [Luviskol | | | | | | |
| K 17]⁵ | | | | | | |
| Salicylic acid | 0.2 | 2 | 1.5 | 1 | 1 | 0.5 |
| Dipropylene glycol | 0 | 12 | 11 | 0 | 11 | 0 |
| Glycerine | 6 | 10 | 9.5 | 12.5 | 3 | 5.0 |
| Carbowax 400 (PEG 8)⁶ | 0.3 | 0.5 | 18 | 0 | 0 | 0 |
| Procetyl AWS⁷ | 3.0 | 5.0 | 0.5 | 3.0 | 3.0 | 3.0 |
| Glycyrrhizic acid | 0 | 0 | 0.1 | 0.5 | 0 | 0 |
| I | | | | | | |
| Deionised water | — | -to- -100- | | — | — | — |
| Na₄EDTA | 0.1 | 0.1 | 0.1 | 0.1 | 0.1 | 0.1 |
| Sodium Citrate | 0.3 | 0.25 | 0.25 | 0.32 | 0.3 | 1.0 |
| Sodium Chloride | 0.3 | 0.3 | 0.3 | 0.3 | 0.3 | 0.3 |
| Citric Acid | 1.0 | 0 | 0.5 | 0 | 1.0 | 0 |
| Methyl Paraben | 0.2 | 0 | 0.1 | 0.05 | 0 | 0 |
| J | | | | | | |
| Zinc Oxide | 0 | 0.4 | 0 | 0 | 0 | 0.45 |

¹Supplied by Dow Corning, Avco House South, Castle S
²Supplied by Procter & Gamble
³Supplied by Cardre Incorporated
⁴Trihydroxystearin, supplied by NL Chemicals, Wycoff Milse Road, Highstown, NJ 08520
⁵Supplied by BASF, Earl Road, Cheadle Hulme, Cheadle, Cheshire, SK8 6QB
⁶Supplied by Union Carbide, 39 Old Ridgebury Road, Danbury
⁷Supplied by Croda Chemicals Ltd., Cowick Hall, Snaith, Goole, North Humberside, DN14 9AA
⁸Supplied by Rose Chemicals
⁹Supplied by Aston Chemicals, Weltech Centre, Ridgeway, Welwyn Garden City, Herts AL7 2AA

The formulations of Examples I to VI can be prepared as follows. The various components listed in the Table have been segregated into groups, the constituents of each group being mixed together before being added to members of the remaining groups in accordance with the procedures set forth below.

In the first step, the mixture of components of phase A is stirred for approximately 15 minutes with shear mixing until homogeneous. With high speed shear mixing, the materials of phase B are added gradually to A nd the batch is mixed for about 30 minutes. Phase C is added and the resulting mixt.,re is ground for approximately 15 minutes.

The components from phase D are then added aid the resulting mixture is ground until fully dispersed.

The waxy phase E is then added to the batch ard the batch is heated to 85° C. with mixing until the waxes have melted and then co iled to 50° C. with stirring. Phase F premix is then added to the batch and homoge, ised for 10 minutes. The batch is cooled to room temperature with stirring. Phase G is added to the batch and homogenised for 10 minutes.

The water phase is prepared as follows. The components of phase I are mixed until dissolved. The components of phase H except for the ethanol are mixed together under high speed shear until dissolved. Phase E is cooled and ethanol is added. The solution is mixed until clear. Phase I is added to phase H and mixed, followed by addition of phase J under mixing.

The water phase is finally added to the oil phase slowly whilst homogenising at a low speed, with stirring. When all of the wate, phase has been added, high shear is applied to the batch for approximately 5 minutes to increase the viscosity of the final product.

The resulting make-up composition is ready for packaging.

The cosmetic compositions of the Examples exhibit anti-acne efficacy, improved product stability and improved salicylic acid sc lubility.

What is claimed is:

1. A cosmetic composition in the form of a water-in-oil emulsion comprising:
(a) continuous oil phase and;
(b) discontinuous aqueous phase comprising:
(i) water
(ii) acidic skin care active which is insoluble in said aqueous phase; and
(iii) nonionic surfactant selected from polyoxyethylene-polyoxypropylene ethers of C4-C22 alcohols, and mixtures thereof.

2. A cosmetic composition according to claim 1 wherein the nonionic surfactant is selected from polyoxyethylene-polyoxypropylene ethers of C4-C22 alcohols having the general formula: 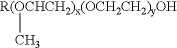
wherein x is in the range of from about 1 to about 35, preferably from about 1 to about 10, y is in the range of from about 1 to about 45, preferably from about 1 to about 30 and R is a straight chain or branched chain C4 to C22 alkyl group, or a mixture thereof.

3. A cosmetic composition according to claim 1 or 2 wherein the nonionic surfactant is selected from a polyoxyethylene-polyoxypropylene ether of cetyl alcohol.

4. A cosmetic composition according to any of claims 1 to 3 wherein the aqueous phase has a pH of about 6 or less, preferably from about 1 to about 5.5, more preferably from about 2 to about 5, and especially from about 2.5 to about 4.

5. A cosmetic composition according to any of claims 1 to 4 wherein the acidic skin care active is selected from salicylic acid, azelaic acid, retinoic acid, lactic acid, glycolic acid, pyruvic acid, and mixtures thereof.

6. A cosmetic composition according to any of claims 1 to 5 wherein the acidic skin care active is salicylic acid.

7. A cosmetic composition according to any of claims 1 to 6 comprising from about 0.1% to about 10%, preferably from about 0.1% to about 5%, more preferably from about 0.5% to about 3%, by weight, of the acidic skin care active.

8. A cosmetic composition according to any of claims 1 to 7 comprising from about 0.1% to about 15%, preferably from about 0.5% to about 10%, more preferably from about 1% to about 5% by weight, of the nonionic surfactant.

9. A cosmetic composition according to any of claims 1 to 8 additionally comprising from about 0.1% to about 30%, preferably from about 0.1% to about 25%, more preferably from about 1% to about 15% by weight of pigment.

10. A cosmetic composition according to claim 9 wherein the pigment is selected from a pigment which has been coated with organosilicon component selected from a polyorganosiloxane and a silane, and mixtures thereof, wherein the coated pigment has a residual hydride content as measured by hydrogen potential of less than about 2.0 ml H²/ g of coated pigment.

11. A cosmetic composition according to claim 10 wherein said polyorganosiloxane of said pigment is selected from
(A) material of the formula:
(R¹ )₃ SiO-(Si(R²R³)0)ₚ-Si(R²R³)OA₂
wherein p is 1 to 1000, preferably from 1 to 100, A₂ is hydrogen or an alkyl group having from 1 to 30 carbon atoms, R¹ is a C₁-C₃₀ alkyl, preferably methyl, R² and R³ are independently selected from a C₁-C₃₀ alkyl and a phenyl, preferably wherein R² and R³ are both methyl or wherein R² is methyl and R³ is phenyl ; or
(B) material of the formula:
(R¹ )₃SiO(Si(R²)(H)-O)i-Si(RI )3 wherein i is I to 1000, preferably from 1 to 100, and wherein R¹ and R² are as defined above for formula (A).

12. A cosmetic composition according to claim 10 wherein the silane is selected from material of the formula:
(C) A₁SiX₁X₂X₃
wherein A is an alkyl or alkenyl group having from 1 to 30 carbon atoms, and X₁, X₂ and X₃ are independently C₁-C₄ alkoxy, preferably methoxy or ethoxy, or halo, preferably chloro.

13. A cosmetic composition according to any of claims 10 to 12 wherein the pigment is selected from iron oxide and titanium dioxide, and mixtures thereof.

14. A cosmetic composition according to any of claims 1 to 13 additionally comprising from about 0.1% to about 10%, preferably from about 0.1% to about 5%, more preferably from about 0.5% to about 2%, by weight, of pyrrolidone-based solubilising agent.

15. A cosmetic composition according to claim 14 wherein the pyrrolidone-based solubilising agent is polyvinylpyrrolidone.

16. A cosmetic composition according to claims 1 to 15 additionally comprising from about 0.01% to about 5%, by weight, of citric acid or salt thereof.

17. A skin care composition according to any of claims 1 to 16 comprising from about 0.01% to about 20%, by weight of the composition, of a liquid, polyol carboxylic acid ester having a polyol moiety and at least 4 carboxylic acid moieties, wherein the polyol moiety is selected from sugars and sugar alcohols containing from about 4 to about 8 hydroxyl groups, and wherein each carboxylic acid moiety has from about 8 to about 22 carbon atoms, and wherein said liquid polyol carboxylic acid ester has a complete melting point of less than about 30° C.

18. A composition according to claim 17 wherein said liquid polyol carboxylic acid ester contains no more than about 2 free hydroxyl groups.

19. A composition according to claim 17 or 18 wherein said carboxylic acid moieties contain from about 14 to about 18 carbon atoms.

20. A composition according to any of claims 17 to 19 wherein said polyol moiety is selected from erythritol, xylitol, sorbitol, glucose, sucrose, and mixtures thereof.

21. A composition according to any of claims 17 to 20 wherein said polyol moiety is sucrose.

22. A composition according to any of claims 17 to 21 wherein said liquid polyol carboxylic acid ester has a complete melting point below about 27.5° C.

23. A composition according to any of claims 17 to 22 wherein said liquid polyol carboxylic acid polyester has a complete melting point below about 25° C.

24. A composition according to any of claims 17 to 23 wherein said liquid carboxylic acid polyol ester is selected from sucrose pentaoleate, sucrose hexaoleate, sucrose heptaoleate, sucrose octaoleate, and mixtures thereof.

25. A cosmetic composition according to any of claims 10 to 13 wherein the coated pigment has a residual hydride content as measured by hydrogen potential of less than about 1.0, preferably less than about 0.5, especially less than about 0.1 ml H²/g of coated pigment.

26. A cosmetic composition according to any of claims 1 to 25 wherein the aqueous phase has a pH of from about 1 to about 5.5, preferably from about 2 to about 5, especially from about 2.5 to about 4.

27. A cosmetic composition according to any of claims 1 to 26 comprising from about 1% to about 50%, by weight, of volatile silicone oil selected from volatile silicones, non-volatile silicones and mixtures thereof.

28. A cosmetic composition according to claim 27 wherein the volatile silicone oil is selected from cyclic polyorganosiloxanes having viscosities of no greater than about 10 centistokes, and linear polyorganosiloxanes having viscosities of less than about 5 centistokes at 25° C., and mixtures thereof.

29. A cosmetic composition according to claim 28 wherein the volatile silicone oil is selected from cyclic polydimethylsiloxanes containing an average of from about 3 to about 9 silicon atoms, preferably from about 4 to about 5 silicon atoms and linear polydimethylsiloxanes containing an average of from about 3 to about 9 silicon atoms.

30. A cosmetic composition according to claim 29 wherein the polydiorganosiloxane-polyoxyalkylene copolymer is dimethicone copolyol.

31. A cosmetic composition according to any of claims 1 to 30 additionally comprising from about 0.1% to about 30%, preferably from about 1% to about 10%, by weight, of humectai ;.

32. A cosmetic composition according to claim 31 wherein the humectant is glycerine.

33. A cosmetic composition according to claim 27 comprising from about 0.1% to about 10%, preferably from a. out 2% to about 5%, by weight, of non-volatile silicones.

34. A cosmetic composition accordin to any of claims 1 to 33 comprising from about 20% to about 95%, prefe ably from about 30% to about 70%, by weight, of the oil phase, and froi i about 5% to about 80%, preferably from about 30% to about 70%, by weigit, of the water phase.
